(19) United States
(12) Patent Application Publication
(10) Pub. No.: US 2020/0170906 A1
(43) Pub. Date: Jun. 4, 2020

(54) HAIR CARE COMPOSITION FOR CLEANSING AND REFRESHING THE HAIR AND THE SCALP

(71) Applicant: The Procter & Gamble Company, Cincinnati, OH (US)

(72) Inventors: Supriya Punyani, Singapore (SG); Ioannis Constantine Constantinides, Wyoming, OH (US); Rahul Vyas, Singapore (SG); Junji Hamano, Singapore (SG); Judith Ann Hollingshead, Batavia, OH (US)

(21) Appl. No.: 16/209,267

(22) Filed: Dec. 4, 2018

(51) Int. Cl. A61K 8/34; A61K 8/36; A61K 8/49; A61K 8/27; A61K 8/25; A61K 8/02; A61Q 5/02

(52) U.S. Cl. CPC A61K 8/345; A61K 8/36; A61K 8/498; A61Q 5/02; A61K 8/25; A61K 8/0275; A61K 8/27

(57) ABSTRACT

The present invention is directed to an aqueous hair care composition for cleansing and refreshing the hair and the scalp comprising a 1,2-diol having a carbon chain with a length of more than 8 carbons in its molecule, hydrophobic particles, and a malodor protection material. The malodor protecting material may be an odor blocking material, an odor masking material or an odor neutralizing material or a combination thereof. The hair composition may be a leave-on or a rinse-off composition.

### FIELD OF THE INVENTION

The present invention relates to a hair care cleansing composition comprising one or more sebum modifiers, that is, materials that can modify one or more sebum physical properties such as melting and/or viscosity of sebum and that absorb sebum, providing durable cleansing of the hair and the scalp. The composition of the invention also comprises one or more malodor protection materials such as odor blocking materials, odor masking material and/or odor neutralizing material, which durably mitigate malodor of sebum and its oxidation products.

### BACKGROUND OF THE INVENTION

Clean Scalp and Hair is described by consumers as having no sticky or greasy feel, no clumped fibers, no odor, and no hair weigh-down. Generally, consumers perceive unclean scalp and hair when sebum in liquid state builds upon their scalp and their hair during the end of the day. The liquid sebum on hair and scalp is often associated with unclean, greasy, oily and dirty look, feel and smell. Sebum is continuously secreted out of the sebaceous glands on scalp in liquid form. Due to dynamic environment (exposure to UV and microflora), sebum is unstable, and its composition rapidly changes. As sebum is composed of lipids such as unsaturated fatty acid, squalene and unsaturated wax esters which are prone to oxidation (air/UV/microflora), it produces characteristic oily and fish-odor smell. The scalp and hair malodor increase as sebum is secreted and oxidized over time. This malodor is one of the judging signal of unclean scalp and hair to consumer during the day and on no-wash day. Most consumers use leave-on with some fragrance which can mask the smell for a short period of time. But the malodor returns soon, sometimes in less than a few minutes. Thus, there is a need for hair care products that can effectively delay the malodor of unclean scalp and hair by changing sebum properties as well as blocking or neutralizing the sebum malodorous compounds.

The present invention has surprisingly found that this can be achieved by the use of a hair care cleansing composition comprising (a) a 1,2-diol, (b) hydrophobic particles, and (c) one or more malodor protection material. Without wishing to be bounded by theory, the presence of the combination of these materials in hair care compositions contribute to the benefits by modifying the sebum physical properties, such as melting characteristic and/or by absorbing the sebum. As a result, the sebum transfer from the scalp to hair fibers is reduced making the scalp and the hair clean and fresh. In addition, the malodor protection materials effectively mitigating sebum-related malodor and eliminate this signal of unclean state of the hair and the scalp. In addition, the hair care compositions of the present invention can be delivered in the form of leave-on treatment without requiring the use of rinsing with water in the shower. This contributes in the convenience of the consumer who can refresh her hair at any location and at any time during the day.

### SUMMARY OF THE INVENTION

The present invention is directed to hair care compositions for cleansing and refreshing the hair and the scalp comprising:
a) from about 0.1 wt. % to about 12 wt. % of a 1,2-diol having a carbon chain with a length of more than 8 carbons;
b) from about 0.1 wt. % to about 10 wt. % of a solid particle, wherein
   (1) the interfacial tension between the solid particle and sebum is from about 5 to about 18 dyn/cm;
   (2) the sebum exhibits spreading coefficient on the solid, which is greater than about 22 dyn/cm; and
   (3) the work of adhesion of the sebum to the solid particle, which is greater than about 75 dyn/cm. from about 0.1 wt. % to about 5 wt. % emulsifier selected from the group consisting of anionic, non-ionic, cationic and amphoteric and mixtures thereof;
c) from about 0.005 wt. % to about 2 wt. % of a malodor protection material; and
d) an aqueous carrier.

The hair care cleansing composition of the present invention provides durable clean, refreshing and malodor protection for the hair and the scalp. It may be used in the shower as a rinse-off product or it can be conveniently used by the consumer outside of the shower as a leave-on product.

### DETAILED DESCRIPTION OF EMBODIMENTS OF THE INVENTION

While the specification concludes with claims which particularly point out and distinctly claim the invention, it is believed the present invention will be better understood from the following description.

The present invention can comprise, consist of, or consist essentially of the essential elements and limitations of the invention described herein, as well any of the additional or optional ingredients, components, or limitations described herein.

All percentages and ratios used herein are by weight of the total composition, unless otherwise designated. All measurements are understood to be made at ambient conditions, where “ambient conditions” means conditions at about 25° C., under about one atmosphere of pressure, and at about 50% relative humidity (RH), unless otherwise designated. All numeric ranges are inclusive of narrower ranges; delineated upper and lower range limits are combinable to create further ranges not explicitly delineated.

The compositions of the present invention can comprise, consist essentially of, or consist of, the essential components as well as optional ingredients described herein. As used herein, “consisting essentially of” means that the composition or component may include additional ingredients, but only if the additional ingredients do not materially alter the basic and novel characteristics of the claimed compositions or methods.

“Apply” or “application” as used in reference to a composition, means to apply or spread the compositions of the present invention onto keratinous tissue such as the hair.

“Dermatologically acceptable” or “cosmetically acceptable” means that the compositions or components described are suitable for use in contact with human skin tissue without undue toxicity, incompatibility, instability, allergic response, and the like. All compositions described herein which have the purpose of being directly applied to keratinous tissue are limited to those being cosmetically acceptable.

“Safe and effective amount” means an amount of a compound or composition sufficient to significantly induce a positive benefit.

“Leave-on,” in reference to compositions, means compositions intended to be applied to and allowed to remain on the keratinous tissue, such as the hair and the scalp. These leave-on compositions are to be distinguished from rinse-off compositions, which are applied to the hair and subsequently (in a few minutes or less) removed either by washing, rinsing, wiping, or the like. Leave-on compositions exclude rinse-off applications such as shampoos, rinse-off conditioners, facial cleansers, hand cleansers, body wash, or body cleansers. The leave-on compositions may be substantially free of cleansing or detersive surfactants. For example, “leave-on” compositions may be left on the keratinous tissue for at least 15 minutes. For example, leave-on compositions may comprise less than 1% detersive surfactants, less than 0.5% detersive surfactants, or 0% detersive surfactants. The compositions may, however, contain emulsifying, dispersing or other processing surfactants that are not intended to provide any significant cleansing benefits when applied topically to the hair.

“Rinse-off” in reference to compositions, means compositions intended to be applied to the hair and/or the scalp, spread and massaged in these substrates, followed by rinsing with water. Alternatively, in reference to the present invention, the rinse-off composition may be removed or partially removed using a substrate.

“Substrate”, as used herein, means a water-insoluble substrate or partially water-insoluble substrate, which comprises single-layer or multi-layer sheet-form materials. Besides paper tissues, corresponding tissue cloths made from fibers or nonwovens may also be used. Examples of natural fibers include silk, cellulose, keratin, wool, cotton, jute, linen, flax; examples of synthetic fibers include acetate, acrylate, cellulose ester, polyamide, polyester, polyolefin, polyvinyl alcohol, polyurethane fibers or even additive-hydrophilized woven polyolefin fabrics and blends of these fibers or woven fabrics.

“Soluble” means at least about 0.1 g of solute dissolves in 100 ml of solvent, at 25° C. and 1 atm of pressure.

All percentages are by weight of the total composition, unless stated otherwise. All ratios are weight ratios, unless specifically stated otherwise. All ranges are inclusive and combinable. The number of significant digits conveys neither a limitation on the indicated amounts nor on the accuracy of the measurements. The term “molecular weight” or “M.Wt.” as used herein refers to the weight average molecular weight unless otherwise stated. The weight average molecular weight may be measured by gel permeation chromatography. “QS” means sufficient quantity for 100%.

The term “substantially free from” or “substantially free of” as used herein means less than about 1%, or less than about 0.8%, or less than about 0.5%, or less than about 0.3%, or about 0%, by total weight of the composition.

“Hair,” as used herein, means mammalian hair including scalp hair, facial hair and body hair, particularly on hair on the human head and scalp.

“Solid Particles”, as used herein, means particle and/or powder blends that may be free flowing compositions or suspensions of synthetic porous agglomerates comprising of organic and/or inorganic compounds.

“Derivatives,” as used herein, includes but is not limited to, amide, ether, ester, amino, carboxyl, acetyl, acid, salt and/or alcohol derivatives of a given compound.

“Polymer,” as used herein, means a chemical formed from the polymerisation of two or more monomers. The term “polymer” as used herein shall include all materials made by the polymerisation of monomers as well as natural polymers. Polymers made from only one type of monomer are called homopolymers. Polymers made from two or more different types of monomers are called copolymers. The distribution of the different monomers can be calculated statistically or block-wise—both possibilities are suitable for the present invention. Except if stated otherwise, the term “polymer” used herein includes any type of polymer including homopolymers and copolymers.

“Malodor protection material” refers to a substance or combination of substances which can mitigate malodor in relation to the hair and the scalp. The malodor protection material may be an “odor blocking” materials an “odor neutralizing’ material or an “odor masking” material.

“Odor blocking material” or “odor neutralizing material” refers to a substance or a combination of substances which can to dull the human sense of smell; such materials (a) may have affinity to malodor detection receptors or (b) may react or interact with malodor compounds to change their chemical and/or physical properties. Non-limiting examples include polyamines, primary amines or secondary amines.

“Odor-masking material” refers to a substance or combination of substances which can mask or hide the effect of a malodorous compound. Odor-masking materials may include a compound or a combination of compounds with a non-offensive or pleasant smell that is dosed so that it limits the ability to sense a malodorous compound. The development of odor-masking compositions may involve the selection of compounds which coordinate with a malodor or an anticipated malodor to change the perception of the overall scent provided by the combination of odorous compounds.

The malodor protection material may include a diluent, such as dipropylene glycol methyl ether, 3-methoxy-3-methyl-3-butanol, and mixtures thereof. The malodor protection material may also include perfume raw materials.

The hair care composition of the present invention can be a leave-on or a rinse-off. It can provide long-lasting clean appearance, clean feel and freshness to the hair and the scalp. well as long-lasting smell. The benefits are achieved by the composition of sebum modifiers and malodor protection materials of the hair care composition. The combination of the sebum modifier and malodor protection material thereof can modify the sebum physical properties such as melting and/or viscosity characteristics, it can absorb sebum and it can mitigate malodor from sebum and from oxidized sebum. The malodor protection benefit related to sebum odor removal is shown by measuring the malodor intensity of sebum on a scalp substrate such as bovine collagen which is treated with hair care composition that contain the inventive components and by comparing it to the malodor intensity from a control scalp substrate that is treated with sebum and the corresponding composition which does not contain the combination of the inventive materials.

The present invention is directed to a hair care composition for cleansing and refreshing the hair and the scalp comprising:
a) from about 0.1 wt. % to about 12 wt. % of a 1,2-diol having a carbon chain with a length of more than 8 carbons;
b) from about 0.1 wt. % to about 10 wt. % of a solid particle, wherein
   (1) the interfacial tension between the solid particle and sebum is from about 5 to about 18 dyn/cm;
   (2) the sebum exhibits spreading coefficient on the solid, which is greater than about 22 dyn/cm; and
   (3) the work of adhesion of the sebum to the solid particle, which is greater than about 75 dyn/cm. from about 0.1 wt. % to about 5 wt. % emulsifier selected from the group consisting of anionic, non-ionic, cationic and amphoteric and
c) from about 0.005 wt. % to about 2 wt. % of a malodor protection material
d) an aqueous carrier.

The combination of the 1,2-diols and solid particles can (a) modify the sebum physical properties such as melting characteristic and (b) absorb the sebum. The hair care cleansing composition can be in the form of a liquid of various viscosity values. If the high shear viscosity is low, it can be delivered as a spray. In all cases, the low shear viscosity of the composition must be relatively high in order to be phase stable, as the composition contains particles that can settle or come to the top.

1. 1,2-Diols (for Modification of the Melting Characteristics of the Sebum

The hair care composition of the present invention may comprise 1,2-diol in a concentration range from about 0.1% to about 12%; from about 0.2 to about 5%; from about 0.5 to about 4%; and from about 1.0% to about 3.0% by weight of the hair care composition. The 1,2-diols in the hair care composition have a carbon chain with length of more than 8 carbons. Non-limiting examples include 1,2-diols include 1,2-dodecanediol, 1,2-decanediol, 1,2-octadecanediol. Without being limited by theory, such 1,2-diols contribute to the modification of the melting characteristics of sebum, making it more available to be absorbed by the solid which is present in the composition.

In an attempt, to identify materials that can modify the physical characteristics of sebum, such as their melting temperature, various mixtures of materials with sebum (in a 1:1 weight ratio) are prepared and their melting characteristics of the mixtures are measured using differential scanning calorimetry (DSC). The DSC method that is used is described in detail in the “Evaluation Methods” section. The results following table provides the corresponding DSC measurements.

TABLE 1
Melting characteristics of mixtures of various substances with sebum

| Sebum | Endothermic | Endothermic | Endothermic | Endothermic |
|---|---|---|---|---|
| Modifier | Temperature | Temperature | Temperature | Temperature |
| Material | Peak 1 (° C.) | Peak 2 (° C.) | Peak 3 (° C.) | Peak 4 (° C.) |
| Petrolatum | −23.00 | 3.00 | 16 | 40 |
| 1,2- | −18.00 | 4.90 | 21 | 36 |
| dodecanediol | | | | |
| Stearyl alcohol | −18.00 | 6.00 | 15 | 36 |
| Cetyl alcohol | −18.00 | 4.00 | 18 | 26 |
| 1,2-Decanediol | −20.00 | | | 42 |
| Stearic acid | −18.20 | 6.50 | | 39 |
| Zinc carbonate | 0.5 | 7 | | 75 |
| Zinc stearate | −4 | 8 | 21 | 97 |
| 1,2- | −4 | 10 | 20 | 51 |
| Octadecanediol | | | | |

2. Hydrophobic Solid Particles (for Absorbing Sebum)

The hair care composition of the present invention may comprise solid particles in a concentration range from about 0.1% to about 10%; from about 0.5% to about 5%; from about 1.0% to about 2.0% by weight of the hair care composition and in a further embodiment from about 1.0 to about 2.0% by weight of the hair care composition.

The particle surface of the particles that are present in the hair care composition have the following properties
(a) the interfacial tension between the solid particle and sebum is from about 5 to about 18 dyn/cm;
(b) the sebum exhibits spreading coefficient on the solid, which is greater than about 22 dyn/cm; and
(c) the work of adhesion of the sebum to the solid particle, which is greater than about 75 dyn/cm.

Non-limiting examples of hydrophobic solid particle or hydrophobically modified solid particle particles that may be present in the hair care composition are silica silylate, zinc carbonate, hydrophobic clay, zinc oxide, polyethylene powders, polypropylene powders, polystyrene powders, calcium silicate, polyethylene, nylon, boron nitride, mica, clays such as bentonite, montmorillonite and kaolin, zeolite, cyclodextrins, fumed silica, synthetic clays such as polymer powders including natural, synthetic, and semisynthetic cellulose, fluorocarbon resins, polypropylene, modified starches of cellulose acetate, particulate cross-linked hydrophobic acrylate or methacrylate copolymers and mixtures thereof. Non-limiting examples of solid particles for the leave-on aqueous cleansing composition are silica silylate, salicylic acid, 2,4-dihydroxy benzoic acid, 4-chlororesorcinol, 1,2,4-Trihydroxybenzene and zinc carbonate.

Other non-limiting examples of hydrophobic solid particle or hydrophobically modified solid particle particles that may be present in the hair care composition include starches hydrophobically modified to have a high capacity for absorbing oils, such starches can be modified with alkyl or alkenyl substituted dicarboxylic acids; such materials may contain counter-ions, for example metals such as aluminum. A non-limiting example of such material is Natrasorb HFB available from National Starch and Chemical Company, U.S.A., which contains aluminum starch octenyl succinate. Other suitable materials from National Starch and Chemical include Natrasorb Bath, Dry-Flow PC, Dry-How XT, and Dry-Flow Pure.

Other non-limiting examples of hydrophobic solid particle or hydrophobically modified solid particle particles that may be present in the hair care composition include modified proteins such as Vegepol (sodium C8-16 isoalkylsuccinyl soy protein succinate) from Brooks Industries, NJ, and the like.

In order to identify solid particles that can effectively absorb sebum, the absorption/adsorption characteristics of sebum on various solid particles are measured using the contact angle method, which is described in the “Evaluation Method” section. The following table provides the corresponding measurements.

TABLE 2
Measurements of sebum absorption/adsorption on various solids

| | | | Total | Work | Spreading | Inter- |
|---|---|---|---|---|---|---|
| | | | (Polar + | of | Co- | facial |
| | | | Non- | Adhesion | efficient | Tension |
| Material | Nonpolar | Polar | polar) | (sebum) | (sebum) | (sebum) |
| Talc | 22.53 | 5.91 | 28.44 | 55.01 | 1.07 | 0.40 |
| Silica | 32.87 | 32.20 | 65.08 | 76.37 | 22.43 | 15.68 |
| Silica | 49.07 | 26.14 | 75.21 | 86.71 | 32.77 | 15.47 |
| silylate | | | | | | |
| (hydro- | | | | | | |
| phobic | | | | | | |
| silica) | | | | | | |
| Cellulose- | 27.50 | 41.00 | 68.50 | 74.24 | 20.30 | 21.23 |
| untreated | | | | | | |
| Zinc | 44.83 | 26.38 | 71.21 | 83.78 | 29.84 | 14.40 |
| Carbonate | | | | | | |

3. Malodor Protection Material

The hair care composition comprises malodor protection materials. A malodor protection material may be a substance or combination of substances which can mitigate malodor in relation to the hair and the scalp. The class of the general malodor protection materials may be further classified is subclasses, such as odor blocking materials, odor neutralizing material, and odor masking materials. The malodor protection material in the hair care composition of the present invention may comprise one substance or a combination of materials from all these subclasses. It can also comprise a diluent or a combination of diluents. Non-limiting examples of diluents include ethyl amyltol, 2-pentenoic acid, dipropylene glycol methyl ether, and 3-methoxy-3methyl-3-butanol, and mixtures thereof.

It can also comprise a perfume raw material or a combination of perfume raw materials, which may solely provide a fragrance.

Odor blocking material or odor neutralizing material refers to a substance or a combination of substances which can to dull the human sense of smell; such materials (a) may have affinity to malodor detection receptors or (b) may react or interact with malodor compounds to change their chemical and/or physical properties. Non-limiting examples include polyamines, primary amines or secondary amines.

Odor-masking material refers to a substance or combination of substances which can mask or hide the effect of a malodorous compound. Odor-masking materials may include a compound or a combination of compounds with a non-offensive or pleasant smell that is dosed so that it limits the ability to sense a malodorous compound. The development of odor-masking compositions may involve the selection of compounds which coordinate with a malodor or an anticipated malodor to change the perception of the overall scent provided by the combination of odorous compounds.

The hair care composition of the present invention may comprise one or more compounds that can be represented by the following structure I:

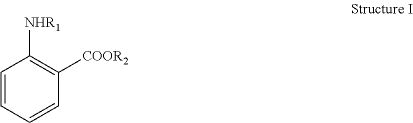

wherein
R₁ can be hydrogen, alkyl group having 1-5 carbon atoms, or acetyl;
R₂ can be methyl, ethyl, propyl, isopropyl, butyl, isobutyl, tert-butyl, pentyl, isopentyl, hexyl, phenethyl, menthyl, or geranyl.

Non-limiting examples of malodor protection material of this type include methyl anthranilate, ethyl anthranilate, propyl anthranilate, isopropyl anthranilate, butyl anthranilate, isobutyl anthranilate, pentyl anthranilate, hexyl anthranilate, heptyl anthranilate, octyl anthranilate, menthyl anthranilate, geranyl anthranilate, phenethyl anthranilate, 2-acetamido benzoate, and dimethyl anthranilate.

The hair care composition of the present invention may also comprise one or more alkyl maltols, such as methyl maltol, ethyl maltol, propyl maltol and mixtures thereof.

The hair care composition of the present invention may also comprise dimethyl-3-(3-methylphenyl)-propanol or 2-methyl 2-pentenoic acid

The hair care composition of the present invention may comprise one or more malodor protection materials in a concentration range from about 0.005% to about 2%; from about 0.01 to about 0.5%; or from about 0.01 to about 0.1% by weight of the hair care composition.

4. Other Components in the Hair Care Composition

The hair care composition may comprise other ingredients.

a. Aqueous Carrier

The hair care composition of the present invention comprises and aqueous carrier. Accordingly, the composition may comprise water from about 78% to about 99.5%; from about 90% to about 99%; or from about 95% to about 98% by weight of the hair care composition. The aqueous carrier may also comprise water miscible solvent or mixture of solvents. Non-limited examples of water-miscible solvents include lower alkyl alcohols and polyhydric alcohols. The lower alkyl alcohols useful herein are monohydric alcohols having 1 to 6 carbons, such as ethanol and isopropanol. The polyhydric alcohols useful herein include propylene glycol, dipropylene glycol, hexylene glycol, glycerin, and propane diol. 1,3-butanediol. The ratio of water-miscible solvent to water may be form about 0 to about 2; from about 0 to about 1; or from about 0 to about 0.8.

b. Rheology Modifier

The hair care composition of the present invention may comprise one or more rheology modifier to provide phase stability and/or improved in-use consumer experience. Any suitable rheology modifier can be used. The leave-on aqueous cleansing composition may comprise from about 0.05% to about 5% of a rheology modifier; from about 0.1% to about 3% of a rheology modifier; or from about 0.5% to about 1% of a rheology modifier by weight of the hair care composition.

The rheology modifier may be a polymeric rheology modifier such as polyacrylamide. The polymeric rheology modifier may be a homopolymer based on acrylic acid, methacrylic acid or other related derivatives. Non-limiting examples include polyacrylate, polymethacrylate, polyethylacrylate, and polyacrylamide. The rheology modifier may also be alkali swellable or hydrophobically-modified alkali swellable acrylic copolymers or methacrylate copolymers non-limiting examples include acrylic acid/acrylonitrogens copolymer, acrylates/steareth-20 itaconate copolymer, acrylates/ceteth-20 itaconate copolymer, acrylates/aminoacrylates copolymer, acrylates/steareth-20 methacrylate copolymer, acrylates/beheneth-25 methacrylate copolymer, acrylates/steareth-20 methacrylate cross-polymer, acrylates/vinyineodecanoate crosspolymer, and acrylates/C10-C30 alkyl acrylate cross-polymer. The rheology modifier may be a soluble cross-linked acrylic polymer; a non-limiting example of this class includes carbomers. The rheology modifier may be a alginic acid-based material, non-limiting examples of which include sodium alginate, and alginic acid propylene glycol esters. The rheology modifier may be an associative polymeric thickeners, non-limiting examples of which include hydrophobically modified cellulose derivatives; hydrophobically modified alkoxylated urethane polymers, nonlimiting example include PEG-150/decyl alcohol/SMDI copolymer, PEG-150/stearyl alcohol/SMDI copolymer, polyurethane-39; hydrophobically modified, alkali swellable emulsions, non-limiting examples include hydrophobically modified polypolyacrylates, hydrophobically modified polyacrylic acids, and hydrophobically modified polyacrylamides; hydrophobically modified polyethers wherein these materials may have a hydrophobe that can be selected from cetyl, stearyl, oleayl, and combinations thereof, and a hydrophilic portion of repeating ethylene oxide groups with repeat units from about 10 to about 300; from about 30 to about 200; from about 40 to about 150. Non-limiting examples of this class include PEG-120-methylglucose dioleate, PEG-(40 or 60) sorbitan tetraoleate, PEG-150 pentaerythrityl tetrastearate, PEG-55 propylene glycol oleate, PEG-150 distearate. The rheology modifier may be cellulose and derivative, non-limiting examples of which include microcrystalline cellulose, carboxymethylcelluloses, hydroxyethylcellulose, hydroxypropylcellulose, hydroxypropylmethylcellulose, methylcellulose, ethyl cellulose, nitro cellulose, cellulose sulfate, cellulose powder, and hydrophobically modified cellulose. The rheology modifier may be a guar or guar derivative, non-limiting examples of which include hydroxypropyl guar, and hydroxypropyl guar hydroxypropyl trimonium chloride. The rheology modifier may be polyethylene oxide; polypropyne oxide or POE-PPO copolymer. The rheology modifier may be polyvinylpyrrolidone, cross-linked polyvinylpyrrolidone or derivative or polyvinyalcohol or its derivative or polyethyleneimine or its derivative. The rheology modifier may be silica, non-limiting examples of which include fumed silica, precipitated silica, and silicone-surface treated silica. The rheology modifier may be water-swellable clay, non-limiting examples of which include laponite, bentolite, montmorilonite, smectite, and hectonite. The rheology modifier may be a gum, non-limiting examples of which include xanthan gum, guar gum, hydroxypropyl guar gum, Arabia gum, tragacanth, galactan, carob gum, karaya gum, and locust bean gum. The rheology modifier may be dibenzylidene sorbitol, karaggenan, pectin, agar, quince seed (Cydonia oblonga Mill), starch (from rice, corn, potato, wheat, etc), starch-derivatives (e.g. carboxymethyl starch, methylhydroxypropyl starch), algae extracts, dextran, succinoglucan, and pulleran,

Non-limiting examples of other rheology modifiers include acrylamide/ammonium acrylate copolymer (and)polyisobutene (and) polysorbate 20, acrylamide/sodium acryloyldimethyl taurate copolymer/isohexadecane/polysorbate 80, acrylates copolymer, acrylates/beheneth-25 methacrylate copolymer, acrylates/C10-C30 alkyl acrylate cross-polymer, acrylates/steareth-20 itaconate copolymer, ammonium polyacrylate/isohexadecane/PEG-40 castor oil, C12-16 alkyl PEG-2 hydroxypropylhydroxyethyl ethylcellulose (HM-EHEC), carbomer, cross-linked polyvinylpyrrolidone (PVP), dibenzylidene sorbitol, hydroxyethyl ethylcellulose (EHEC), hydroxypropyl methylcellulose (HPMC), hydroxypropyl methylcellulose (HPMC), hydroxypropylcellulose (HPC), methylcellulose (MC), methylhydroxyethyl cellulose (MEHEC), PEG-150/decyl alcohol/SMDI copolymer, PEG-150/stearyl alcohol/SMDI copolymer, polyacrylamide/C13-14 isoparaffin/laureth-7, polyacrylate 13/polyisobutene/polysorbate 20, polyacrylate crosspolymer-6, polyamide-3, polyquaternium-37 (and) hydrogenated polydecene (and) trideceth-6, polyurethane-39, sodium acrylate/acryloyldimethyltaurate/dimethylacrylamide, crosspolymer (and) isohexadecane (and) polysorbate 60, sodium polyacrylate. Exemplary commercially-available rheology modifiers include ACULYN™ 28, Klucel M CS, Klucel H CS, Klucel G CS, SYLVACLEAR AF1900V, SYLVACLEAR PA1200V, Benecel E10M, Benecel K35M, Optasense RMC70, ACULYN™33, ACULYN™46, ACULYN™22, ACULYN™44, Carbopol Ultrez 20, Carbopol Ultrez 21, Carbopol Ultrez 10, Carbopol Ulterez 30, Carbopol 1342, Sepigel™ 305, Simulgel™600, Sepimax Zen, and combinations thereof.

c. Structurants

The hair care composition of the present invention may comprise structurants or suspending agents including crystalline suspending agents which can be categorized as acyl derivatives, long chain amine oxides, and mixtures thereof. These suspending agents are described in U.S. Pat. No. 4,741,855. These suspending agents include ethylene glycol esters of fatty acids in one aspect having from about 16 to about 22 carbon atoms. In one aspect, useful suspending agents include ethylene glycol stearates, both mono and distearate, but in one aspect, the distearate containing less than about 7% of the mono stearate. Other suitable suspending agents include alkanol amides of fatty acids, having from about 16 to about 22 carbon atoms, or even about 16 to 18 carbon atoms, examples of which include stearic monoethanolamide, stearic diethanolamide, stearic monoisopropanolamide and stearic monoethanolamide stearate. Other long chain acyl derivatives include long chain esters of long chain fatty acids (e.g., stearyl stearate, cetyl palmitate, etc.); long chain esters of long chain alkanol amides (e.g., stearamide diethanolamide distearate, stearamide monoethanolamide stearate); and glyceryl esters (e.g., glyceryl distearate, trihydroxystearin, tribehenin) a commercial example of which is Thixin® R available from Rheox, Inc. Long chain acyl derivatives, ethylene glycol esters of long chain carboxylic acids, long chain amine oxides, and alkanol amides of long chain carboxylic acids in addition to the materials listed above may be used as suspending agents. Other long chain acyl derivatives suitable for use as suspending agents include N,N-dihydrocarbyl amido benzoic acid and soluble salts thereof (e.g., Na, K), particularly N,N-di(hydrogenated) C16, C18 and tallow amido benzoic acid species of this family, which are commercially available from Stepan Company (Northfield, Ill., USA). Examples of suitable long chain amine oxides for use as suspending agents include alkyl dimethyl amine oxides, e.g., stearyl dimethyl amine oxide. Other suitable suspending agents include primary amines having a fatty alkyl moiety having at least about 16 carbon atoms, examples of which include palmitamine or stearamine, and secondary amines having two fatty alkyl moieties each having at least about 12 carbon atoms, examples of which include dipalmitoylamine or di(hydrogenated tallow)amine. Still other suitable suspending agents include di(hydrogenated tallow)phthalic acid amide, and crosslinked maleic anhydride-methyl vinyl ether copolymer.

d. Emulsifiers

The 1,2-diol may be added into the hair care composition neat or as pre-emulsion. In the latter case, an emulsifier is used to make the pre-emulsion. The emulsifier selection is guided by the Hydrophilic-Lipophilic-Balance value (HLB value) of emulsifiers. Suitable range of HLB value is from about 6 to about 16; or from about 8 to about 14. Emulsifiers with an HLB higher than 10 are water soluble. Emulsifiers with low HLB are lipid soluble. To obtain suitable HLB value, a mixture of two or more emulsifiers may be used. Suitable emulsifiers include non-ionic, cationic, anionic and amphoteric emulsifiers. The concentration of the emulsifier in the composition should be sufficient to provide the desired emulsification of the conditioning actives to achieve desired particle sizes and emulsion stability, and generally ranges from about 0.1 wt % to about 5 wt. %, from about 0.5 wt. % to about 3 wt. %, from about 0.6 wt. % to about 2 wt. % by weight of the composition.

e. Solvents

The hair care composition of the present invention may comprise one or more solvents that are not water-miscible selected from the group consisting of isopropyl myristate, 2-hexyldecanol, PEG-3 glyceryl Cocoate, PEG-7 glyceryl cocoate, isododecane and isoparaffin C11-12 and mixtures thereof. The concentration of the solvent in the composition may range from about 0.1 wt % to about 20 wt. %, from about 0.5 wt. % to about 10 wt. %, from about 1 wt % to about 5 wt. % by weight of the composition.

f. Detersive Surfactants

The hair care composition of the present invention may also comprise one or more detersive surfactants. The detersive surfactants may be anionic, amphoteric, nonionic, cationic or mixtures thereof. The concentration of the detersive surfactant in the composition may range from about 0.1 wt % to about 10 wt. %, from about 0.5 wt. % to about 10 wt. %, from about 1 wt. % to about 5 wt. % by weight of the composition.

g. pH of the Composition

The hair care composition of the present invention may also comprise one or more pH adjusting material. The composition may have a pH in the range from about 2 to about 10, at 25° C.; in the range from about 2 to about 7; or in the range of 3.5 to about 6.5. Alternatively, the composition may have a pH in the range from about 3.5 to 6 or in the range from about 5.25 to about 7. The hair care composition of the present invention may further comprise one or more pH buffering agent. Examples of buffering agents are well known in the art and include, for example, ammonia/ammonium acetate mixture and monoethanolamine (MEA), citric acid and citrate salt.

h. Conditioning Benefit Agents

The hair care composition of the present invention may also comprise one or more conditioning benefit agent, such as silicones or organic hydrophobic oils. The conditioning agents that are oils should be added in moderate quantities, for example, less than 3 wt % by weight of the hair care composition, in order to preserve the absorbing ability of the solid particles of the composition. The concentration of the conditioning benefit in the composition may range from about 0 wt % to about 3 wt. %, from about 0 wt. % to about 1 wt. %, from about 0 wt. % to about 0.5 wt. % by weight of the composition.

i. Other Ingredients

The hair care composition of the present invention may also comprise one or more other benefit agents or other ingredients such anti-dandruff agents, anti-itch agents, other scalp health agents, anti-oxidants, vitamins, chelation agents, sensates, colorants, bleaching agents, preservatives, perfumes, humectants and mixtures thereof.

Particularly important is the presence of perfumes and/or sensates. Such ingredients may provide sensorial signals to the consumer during and after the cleansing method that the hair and/or the scalp is clean and refreshed.

### Product Forms

The hair care composition of the present invention can be a leave-on or a rinse-off composition. It can be delivered as a spray or a liquid or a foam directly on the hair and/or the scalp or it can be delivered on the hands and then applied onto the hair or the scalp. It can also be delivered on a substrate, such as a nonwoven and then applied on the hair and/or the scalp. sprayed on the substrate and the applied to the hair and scalp. As mentioned above, it can be rinsed off with water after the application immediately after its application or a few minute after its application. It can be also left of the hair or the scalp after its application for 15 minutes or longer periods of time (leave-on). In addition, a substrate, such as a nonwoven, can be used to partially remove the composition form the hair or the scalp any time after its application.

### Evaluation Methods

A. Sample Preparation for Differential Scanning Calorimetry (DSC) Measurement

An amount of 1.0 g of artificial sebum (Refer table 3 for artificial sebum composition) is mixed with 1.0 g of sebum modifier material at room temperature and heated at 40° C. water bath for 2 minutes to a uniform solution/mixture and the solution/mixture is cooled at room temperature. The melting characteristics of the prepared sample are determined using the differential scanning calorimetry (DSC) method as described below.

TABLE 3
Artificial Sebum Composition.

| Raw Material | Weight % |
|---|---|
| Stearic Acid | 14 |
| Oleic acid | 8 |
| Squalene | 12 |
| Cetyl Palmitate | 12 |
| Isostearyl Isostearate | 12 |
| Trioctanoin | 20 |
| Caprylic Capric | 20 |
| Triglyceride | |
| Cholesterol | 2 |

B. Differential Scanning Calorimetry (DSC) Measurement

A DSC 204 Netzsch TASC 414/3A is used for the tests, which are performed in triplicate samples. The samples consist of approximately 5.5 mg of a 1:1 uniform solution/mixture combination of artificial sebum and sebum modifier. The sample is placed into T-Zero aluminum DSC pans, and then covered with stainless steel mesh A sand baseline and burn-off is performed before running samples, and after every 10 samples. The tests are conducted within the temperature range of −50° C. to 300° C. at 5° C./min under 200 ml/min nitrogen purge. An empty pan of the same type is employed as a reference and tested under the same experimental conditions. The standard deviation of this method is less than 5%. Temperature measurements are taken at the endotherm peaks of physical changes in sebum components and curve-fitting energy integration is performed for the entire endotherm.

C. Measurement of Solid Particles Physical Properties Using Contact Angle Method:

The contact angle on solid particles is determined using a modified ASTM D7490-13, titled “Standard Test Method for Measurement of the Surface Tension of Solid Coatings, Substrates and Pigments using Contact Angle Measurements”.

The contact angle on both sides of a drop of distilled water is measured on the finished side of the clean untreated ceramic tile. The contact angle measurement is repeated for a drop of diidomethane (supplied by Sigma Aldrich, St. Louis, Mo.). The two contact angle values (for water and diidomethane) are then substituted into two separate expressions of the Owens-Wendt-Kaelble equation (one for each liquid). This results in three equations and two unknowns, which are then solved for the dispersion and polar components of surface tension.

Equipment Used for Contact Angle Method

Goniometer—An instrument consisting of a controlled light source, a stage to hold the tile, and a microscope or camera for viewing of the drop on the tile is required (First Ten Angstrom, Model 200, or equivalent).
Hypodermic Syringe—A gas tight syringe is used, such as a 1-mL hypodermic syringe, equipped with a No. 27 blunt tipped stainless steel needle, capable of providing 100 to 200 drops from 1 mL.

Reagents and Materials

Water—Type II reagent water (distilled) in accordance with ASTM Specification D1193-99. Diiodomethane (99+% purity).

Procedure

The tile is tested in a constant temperature (73° F.±2° F.) and humidity environment (50±5% relative humidity). The goniometer is set up and the stage is leveled according to the manufacturer's instructions. The contact angle is measured for each discrete droplet of water and diiodomethane on the tile as described in ASTM D7334 or the manufacturer's literature for the instrument being used. The tile is positioned to deposit a drop without visible distortion of the drop shape due to movement. The tip of the hypodermic needle is set at the distance from the surface recommended by the manufacturer of the instrument (3 mm (⅛ in.) and a drop of test liquid 5 μL in size is deposited on the tile. The drop size is controlled to ±0.1 μL.

Contact Angle

The camera or video device is focused so that the image of the drop can be captured. Two measurements are made (one on each drop edge) for each of two drops (water and diiodomethane) on the tile using commercial software designed to extract contact angles from movies or images. For example, First Ten Angstrom software version 2.1, build 363, can be used or equivalent. If the contact angles on two edges are different by more than 4°, the values are eliminated and the test is repeated. The measurement is repeated 5 more times on new droplets. The contact angle for the tile is reported as the average of the six angles measured for each side.

High Speed Video Imaging.

The image acquisition speed captures at least 10-20 images from the time the drop hits the surface to the time it cannot be resolved from the surface of the sample. A capture rate of 900 images/s is used. The software described above extracts the contact angles from the video feed. The droplet volume is also calculated using the same software under the sessile volume. The contact angles are plotted with the sessile volume plots. Enough time is allowed for the drop to wet out to equilibrium. However, in highly absorptive systems, the drop absorbs into the material before equilibrium is achieved. In such cases in which the drop rapidly (<0.2 s) absorbs into the substrate, the video is progressed until 2% of the volume of the drop absorbs into the substrate. The contact angle is recorded at that time point. This might mean that the first resolved image in extremely fast absorbing systems if the second image shows more than 2% volume loss.

Surface Energy Method

Calculation

The Owens-Wendt-Kaelble equation:

$${σ}_{lg}^{T} \frac{( cos θ + 1 )}{2} = {( {σ}_{lg}^{D} {γ}_{sg}^{D} )}^{1 / 2} + {( {σ}_{lg}^{P } {γ}_{sg}^{P} )}^{1 / 2}$$

where:
θ=the average contact angle for the test liquid on the test specimen,
σ_{lg}^{T}=the total surface tension of the test liquid in dyn/cm
σ^{D} and σ^{P}=the dispersive and polar components of the liquid surface tension, respectively, also in dyn/cm.
σ_{sg}=the total surface energy of the test substrate in dyn/cm
σ^{D} and σ^{P}=the dispersive and polar components of the test substrate, respectively, also in dyn/cm.

| | Surface Tension (σ_{lg}) (dyn/cm) | | |
|---|---|---|---|
| Solvent | Nonpolar | Polar | Total |
| Diiodomethane | 50.8 | 0 | 50.8 |
| Water | 21.8 | 51.0 | 72.8 |

The Owens-Wendt-Kaelble equation is simplified to the following when a dispersive (nonpolar) solvent such as diiodomethane is used:

$${σ}_{lg}^{T} \frac{( cos θ + 1 )}{2} = {( {σ}_{lg}^{D} {γ}_{sg}^{D} )}^{1 / 2}$$

The dispersive (nonpolar) component of surface energy (σ^{D}_{sg}) is determined. Surface tension properties for diiodomethane are known and included in the table above. The contact angle is experimentally determined using the method delineated above.

Upon inserting the calculated dispersive component of surface energy (σ^{D}_{sg}) for the substrate into the Owens-Wendt-Kaelble equation delineated above and using the contact angles determined for water, the polar component of surface energy (σ^{P}_{sg}) of the substrate is determined because the surface tension properties for water are known and included in the table above. The dispersive component (σ^{D}_{sg}) of the substrate is determined with diiodomethane as explained above.

Thermodynamic Parameters

Thermodynamic parameters are calculated by inserting surface energy components into the following equations of state.

Spreading Coefficient:

The spreading coefficient (S) is determined by de Gennes:

S=γ_{sg}^{T}−σ_{lg}^{T}−σₛₗ

where σₛₗ is the interfacial tension
Reference: de Gennes, P.-G., Reviews of Modern Physics (1985), 57, 827-863 Interfacial Tension:
   The Owens-Wendt equation of state is used to determine interfacial tension (σₛₗ):

σₛₗ^{T}=γ_{sg}^{T}+σ_{lg}^{T}−2(σ_{lg}^{D}γ_{sg}^{D})^{1/2}−2(σ_{lg}^{P}γ_{sg}^{P})^{1/2}
Reference: D. K. Owens and R. C. Wendt, Journal of Applied Polymer Science (1969), 13, 1741-1747.

Work of Adhesion:

The work of adhesion (W) using the Dupre equation of state:

W=γ_{sg}^{T}+σ_{lg}^{T}−σₛₗ
Reference: A. Dupré, Theorie Mechanique de la Chaleur; Gauthier-Villars: Paris, 1869; pp 36W.

D. Method of Determination of Odor

### Method of Preparing Sebum-Treated Substrate Samples

An amount of 10 μg of artificial sebum is spread on a bovine collagen skin (5 cm×5 cm in dimension), using a finger glove. The sebum is spread on the bovine collagen skin so that the skin surface is completely covered with the sebum. Then, the skin is stored and covered in a petri dish. For each data point, three repeat experiments are performed. An amount of 10 μg of the hair care composition is applied on the sebum-treated collagen skin and the skin is again covered with a petri dish.

### Determination of Sebum Odor

The Bovine Collagen skin which is treated with sebum is evaluated for odor via a sensory evaluation. The sensory evaluation of sebum treated collagen is done with the help of three malodor expert graders (n=3) and the data are averaged. The evaluation scale used is 0-100:
0 being no odor present
10 being very slight odor
20 slight odor “I think there is an odor present”
25 slight odor “I detect something but cannot identify specific odor”

### 50 Moderate

75 Strong odor
100 Extremely strong odor

For each experiment (data point) 3 samples are prepared, evaluated and averaged.

E. Method of Determination of Sebum Removal

### Method of Preparing Sebum-Fluorescence Dye

An amount of 20 g of artificial sebum is mixed with 0.03 g of Tinopal B (Benzoxazole, 2,2′-(2,5-thiophenediyl) bis[5-(1,1-dimethylethyl)], from BASF). The mixture is prepared in a brown vial to prevent light exposure and heated to 54° C. using water bath to melt and mix the components. Artificial Sebum Composition is prepared by adding materials mentioned in Table 3 and then heated to 54° C. using water bath, to make it a uniform mixture.

Method of Treating Hair with Aqueous Pre-Wash Composition

An amount of 0.20 g of the sebum-fluorescence dye mixture is applied and massaged onto hair switch onto natural virgin brown hair switches weighing 4.0 g via a syringe (dosage 0.05 g of sebum-fluorescence per g of hair). Immediately afterwards, an image of the hair switch is acquired using a digital single-lens reflex camera with parallel polarizers (image at to) under 256 nm UV light having power of 8 W. A quantity of 0.4 g of the aqueous pre-wash composition is then applied, spread on hair switch and is left on the hair switch under 25 oC and 50% relative humidity for 30 minutes. Then, the hair is wetted with water and 0.4 g of shampoo is applied (dosage 0.1 g of shampoo per g of hair). The shampoo is massaged into the hair for 10 seconds and rinsed with deionized water for 10 seconds at a flow rate of 20 ml/minute. The hair Switch is then allowed to air dry and images are taken using the same camera under the conditions described above (image at t_{w}). The hair switch in this case is also assessed by expert graders, as described below. The area of the image occupied by blue color intensity light due to sebum fluorescence mixture is analyzed (selecting the entire hair switch) using 2D projection. For this analysis Java-based image processing program is used. Then, the mean projected area is determined for the hair switch at to (At₀) and for the hair at t_{w} (At_{w}) and the sebum removal is calculated using the equation given below. Each experiment is repeated with three hair switches and the results are averaged. The percent sebum removal is calculated using below equation: % Sebum Removal=100× (At_{w}/At₀). The standard error of sebum removal is less than 10%.

F. Evaluation of Clean Feel and Clean Appearance

### Hair Switch Clean Appearance and Feel Assessment Method

The air dried treated hair switches are rated by ten expert graders in terms of clean (non-greasy) appearance and feel based on a 5-point scale, 5 being the best clean (no greasy) and 1 being the worst clean (very greasy).

### Examples and Compositions

The following examples illustrate embodiments of the invention described herein. The exemplified hair care compositions can be prepared by conventional formulation and mixing techniques. It will be appreciated that other modifications of the hair care compositions within the skill of those in the formulation art can be undertaken without departing from the spirit and scope of this invention. All parts, percentages, and ratios herein are by weight unless otherwise specified. Some components may come from suppliers as dilute solutions. The amount stated reflects the weight percent of the active material, unless otherwise specified.

### Methods of Making the Compositions

The 1,2-diol and solid particles may be added into the hair care composition neat or as pre-emulsion. In the latter case, an emulsifier is used to make the pre-emulsion. The compositions exemplified in the example tables, the 1,2-diol(s) is added into the hair care composition as a pre-emulsion. The pre-emulsion making process is the making of step a below.

A non-limiting example of a method of making the leave-on aqueous cleansing composition comprises the following steps:
a. Mixing all oil-soluble components (including the 1,2-diol and solid particle) in a vessel.

Heating of the components may be needed in order to allow the components to be melted;
b. Mixing all water-soluble components in a separate vessel, adjusting the temperature of the contents of the vessel to the same temperature as the oil phase of step a;
c. Combining the oil phase and the aqueous phase under a high shear mixer, such as a Turrax mixer (supplied by IKA);
d. Adding the thickener/rheology modifier (Sepigel 305 in some of the examples), if required, into the mixture of step c, applying high-speed mixing for 2-5 minutes until a uniform mixture is obtained.

Composition Examples Odor Removal

TABLE 4
Hair Care Compositions for Cleansing and Refreshing the Hair and the Scalp

| | No | Control | Comparative | Comparative | |
|---|---|---|---|---|---|
| | Treatment | Composition | Ex I | Ex II | |
| Ingredient | Wt % | Wt % | Wt % | Wt % | |
| Distilled Water | QS | QS | QS | QS | |
| Ethanol | 0 | 50 | 50 | 50 | |
| Polyacrylamide & C13-14 | 0 | 0.75 | 0.75 | 0.75 | |
| Isoparaffin & Laureth-7 | | | | | |
| (Sepigel 305) | | | | | |
| Perfume | 0 | 0.2 | 0.2 | 0.2 | |
| 1,2-Decanediol | 0 | 0 | 2 | 0 | |
| Zinc carbonate | 0 | 0 | 0.5 | 0 | |
| Salicylic acid | 0 | 0 | 0 | 0 | |
| Malodor Protection Material 1 (a) | 0 | 0 | 0 | 0 | |
| Malodor Protection Material 2 (b) | 0 | 0 | 0 | 0.01 | |
| Mixture of esters, alcohol, | 0 | 0 | 0 | 0 | |
| aldehyde (c) | | | | | |
| Laureth-7 (emulsifier) | 0 | 0.2 | 0.2 | 0.2 | |
| PEG-100 Stearate (emulsifier) | 0 | 0.2 | 0.2 | 0.2 | |
| Cetearyl glucoside & Cetearyl | 0 | 0.2 | 0.2 | 0.2 | |
| alcohol | | | | | |
| Methyl paraben | 0 | 0.2 | 0.2 | 0.2 | |
| Propyl paraben | 0 | 0.15 | 0.15 | 0.15 | |
| Benzyl alcohol | 0 | 0.4 | 0.4 | 0.4 | |
| Phenoxyethanol | 0 | 0.4 | 0.4 | 0.4 | |
| Odor rating | 40 | 30 | 25 | 10 | |
| 0 is no odor and 100 is strong | | | | | |
| odor | | | | | |
| | | Comparative | Comparative | Comparative | Comparative |
| | Ex III | Ex IV | Ex V | Ex VI | Ex VII |
| Ingredient | Wt % | Wt % | Wt % | Wt % | Wt % |
| Distilled Water | QS | QS | QS | QS | QS |
| Ethanol | 50 | 50 | 50 | 50 | 50 |
| Polyacrylamide & C13- | 0.75 | 0.75 | 0.75 | 0.75 | 0.75 |
| 14 Isoparaffin & | | | | | |
| Laureth-7 | | | | | |
| (Sepigel 305) | | | | | |
| Perfume | 0.2 | 0.2 | 0.2 | 0.2 | 0.2 |
| 1,2-Decanediol | 2 | 0 | 0 | 0 | 0 |
| Zinc carbonate | 0.5 | 0 | 0 | 0 | 0 |
| Salicylic acid | 0 | 2 | 0 | 1.95 | 0 |
| Malodor Protection | 0 | 0 | 0.05 | 0.05 | 0 |
| Material 1 (a) | | | | | |
| Malodor Protection | 0.01 | 0 | 0 | 0 | 0 |
| Material 2 (b) | | | | | |
| Mixture of esters, | 0 | 0 | 0 | 0 | 0.01 |
| alcohol, aldehyde (c) | | | | | |
| Laureth-7 (emulsifier) | 0.2 | 0.2 | 0.2 | 0.2 | 0.2 |
| PEG-100 Stearate | 0.2 | 0.2 | 0.2 | 0.2 | 0.2 |
| (emulsifier) | | | | | |
| Cetearyl glucoside & | 0.2 | 0.2 | 0.2 | 0.2 | 0.2 |
| Cetearyl alcohol | | | | | |
| Methyl paraben | 0.2 | 0.2 | 0.2 | 0.2 | 0.2 |
| Propyl paraben | 0.15 | 0.15 | 0.15 | 0.15 | 0.15 |
| Benzyl alcohol | 0.4 | 0.4 | 0.4 | 0.4 | 0.4 |
| Phenoxyethanol | 0.4 | 0.4 | 0.4 | 0.4 | 0.4 |
| Odor rating | 5 | 25 | 25 | 20 | 30 |
| 0 is no odor and 100 is | | | | | |
| strong odor | | | | | |

(a) Mixture of methyl anthranilate or isobutyl anthranilate, 2,2-dimethyl-3-(3-methylphenyl)-propanol with ethers and acetates.
(b) Mixture ethyl maltol or methyl maltol, 2-methyl 2-pentenoic acid with esters, alcohol, aldehyde.
(c) Same mixture of esters, alcohol, aldehyde as in (b) above.

Results:

Composition of inventive Ex III, which comprises all the inventive elements, 1,2-diol, hydrophobic solid particles and a Malodor Protection Material, to the Control and Comparative Examples, shows that the inventive example provides significant reduction in odor.

Composition Examples—Cleansing Performance

### Pre-Wash Compositions

TABLE 5
Examples of Cleansing Compositions

| | Examples | | | |
|---|---|---|---|---|
| | | Comparative | Comparative | Comparative |
| | Control | Ex A | Ex B | Ex C |
| Ingredient | wt % | wt % | wt % | wt % |
| Distilled water | QS | QS | QS | QS |
| Polyacrylamide & C13-14 | 0.75 | 0.75 | 0.75 | 0.75 |
| Isoparaffin & Laureth-7 (Sepigel | | | | |
| 305) | | | | |
| Perfume | 0.2 | 0.2 | 0.2 | 0.2 |
| 1,2-Decanediol | 0 | 1 | 0 | 0 |
| Cellulose | 0 | 0 | 1 | 0 |
| Zinc carbonate | 0 | 0 | 0 | 1.6 |
| Hydrophobic silica | 0 | 0 | 0 | 0 |
| 1,2-Dodecanediol | 0 | 0 | 0 | 0 |
| Isododecane | 0 | 0 | 0 | 0 |
| Laureth-7 (emulsifier) | 0.2 | 0.2 | 0.2 | 0.2 |
| PEG-100 stearate (emulsifier) | 0.2 | 0.2 | 0.2 | 0.2 |
| Cetearyl glucoside & Cetearyl | 0.2 | 0.2 | 0.2 | 0.2 |
| alcohol | | | | |
| Methyl paraben | 0.2 | 0.2 | 0.2 | 0.2 |
| Propyl paraben | 0.15 | 0.15 | 0.15 | 0.15 |
| Benzyl alcohol | 0.4 | 0.4 | 0.4 | 0.4 |
| Phenoxyethanol | 0.4 | 0.4 | 0.4 | 0.4 |
| % Sebum Removed on Hair. | 78 | 90 | 72 | 81 |
| Product dose of 0.05 g of | | | | |
| composition for 1.0 g of hair | | | | |
| Clean Feel Rating at dose of | 2 | 3.5 | 2 | 3 |
| 0.10 g of composition for 1.0 g of | | | | |
| hair | | | | |
| (5 scale rating; 5 is the highest | | | | |
| clean feel and 1 is the lowest) | | | | |
| Clean Look Rating at dose of | 2 | 3.8 | 2.5 | 3 |
| 0.10 g of composition for 1.0 g of | | | | |
| hair | | | | |
| (5 scale rating; 5 is the highest | | | | |
| clean look and 1 is the lowest) | | | | |
| | Examples | | | |
| | Comparative | Comparative | Comparative | |
| | Ex D | Ex E | Ex. F | Ex G |
| Ingredient | wt % | wt % | wt % | wt % |
| Distilled water | QS | QS | QS | QS |
| Polyacrylamide & C13-14 | 0.75 | 0.75 | 0.75 | 0.75 |
| Isoparaffin & Laureth-7 | | | | |
| (Sepigel 305) | | | | |
| Perfume | 0.2 | 0.2 | 0.2 | 0.2 |
| 1,2-Decanediol | 0 | 0 | 1 | 1 |
| Cellulose | 0 | 0 | 1 | 0 |
| Zinc carbonate | 0 | 0 | 0 | 0 |
| Hydrophobic silica | 1 | 0 | 0 | 1 |
| 1,2-Dodecanediol | 0 | 1 | 0 | 0 |
| Isododecane | 0 | 0 | 0 | 1 |
| Laureth-7 | 0.2 | 0.2 | 0.2 | 0.2 |
| PEG-100 stearate | 0.2 | 0.2 | 0.2 | 0.2 |
| Cetearyl glucoside & Cetearyl | 0.2 | 0.2 | 0.2 | 0.2 |
| alcohol | | | | |
| Methyl paraben | 0.2 | 0.2 | 0.2 | 0.2 |
| Propyl paraben | 0.15 | 0.15 | 0.15 | 0.15 |
| Benzyl alcohol | 0.4 | 0.4 | 0.4 | 0.4 |
| Phenoxyethanol | 0.4 | 0.4 | 0.4 | 0.4 |
| % Sebum Removed on Hair. | 86 | 85 | 76 | 97 |
| Product dose of 0.05 g of | | | | |
| composition for 1.0 g of hair | | | | |
| Clean Feel Rating at dose of | 3.5 | 3 | 3.5 | 4.5 |
| 0.10 g of composition for 1.0 g | | | | |
| of hair | | | | |
| (5 scale rating; 5 is the highest | | | | |
| clean feel and 1 is the lowest) | | | | |
| Clean Look Rating at dose of | 3.5 | 3 | 3.5 | 4.5 |
| 0.10 g of composition for 1.0 g | | | | |
| of hair | | | | |
| (5 scale rating; 5 is the highest | | | | |
| clean look and 1 is the lowest) | | | | |
| | Examples | | | |
| | Ex. H | Ex. i | Comparative | Comparative |
| | (wt./wt.) | (wt./ | Example K | Example L |
| Ingredient | % | wt.) % | (wt./wt.) % | (wt./wt.) % |
| Distilled water | QS | QS | QS | QS |
| Polyacrylamide & C1344 | 0.75 | 0.75 | 0.75 | 0.75 |
| Isoparaffin & Laureth-7 (Sepigel | | | | |
| 305) | | | | |
| Perfume | 0.2 | 0.2 | 0.2 | 0.2 |
| 1,2-Decanediol | 1 | 0 | 0 | 1.0 |
| Zinc carbonate | 0 | 1.6 | 0 | 0 |
| Hydrophobic silica | 1 | 0 | 0 | 0 |
| 1,2-Dodecanediol | 0 | 1.0 | 1.0 | 0 |
| Talc | 0 | 0 | 1.0 | 1.0 |
| Laureth-7 | 0.2 | 0.2 | 0.2 | 0.2 |
| PEG-100 stearate | 0.2 | 0.2 | 0.2 | 0.2 |
| Cetearyl glucoside & Cetearyl | 0.2 | 0.2 | 0.2 | 0.2 |
| alcohol | | | | |
| Methyl paraben | 0.2 | 0.2 | 0.2 | 0.2 |
| Propyl paraben | 0.15 | 0.15 | 0.15 | 0.15 |
| Benzyl alcohol | 0.40 | 0.40 | 0.40 | 0.40 |
| Phenoxyethanol | 0.40 | 0.40 | 0.40 | 0.40 |
| % Sebum Removed on Hair. | 93 | 90 | 68 | 76 |
| Product dose of 0.05 g of | | | | |
| composition for 1.0 g of hair | | | | |
| Clean Feel Rating at dose of 0.10 | 4.0 | 3.5 | 1.0 | 2.0 |
| g of composition for 1.0 g of hair | | | | |
| (5 scale rating; 5 is the highest | | | | |
| clean feel and 1 is the lowest) | | | | |
| Clean Look Rating at dose of 0.10 | 3.8 | 3.8 | 3.2 | 2.0 |
| g of composition for 1.0 g of hair | | | | |
| (5 scale rating; 5 is the highest | | | | |
| clean look and 1 is the lowest) | | | | |

Results:

Compositions of Ex. A, Ex. D, Ex. E, Ex. G, Ex. H, Ex. i showed increase in % sebum removal than control prewash treatment. The feel assessment results indicate that combinations of

(a) 1,2-decanediol and zinc carbonate;

(b) 1,2-decanediol and hydrophobic silica; provide, not only sebum removal (resulting in clean benefit), but also clean feel benefit. This is shown by the feel comparison of (a) Ex. G versus Ex. A and Ex. C, and (b) Ex. H versus Example A and C. Thus, it is shown that a combination of a 1,2-diol and hydrophobic particles provide improved sebum cleansing, clean feel and clean look.

The formulations of the present invention may be present in typical hair care compositions. They may be in the form of dispersion, emulsions, powders, talcs, encapsulated, spheres, spongers, solid dosage forms, foams, and other delivery mechanisms. The composition of the present invention may be hair tonics, leave-on hair products, rinse-off hair products such as conditioners, treatment, and styling products, and any other form that may be applied to the hair.

In the examples, all concentrations are listed as weight percent, unless otherwise specified and may exclude minor materials such as diluents, filler, and so forth. The listed formulations, therefore, comprise the listed components and any minor materials associated with such components. As is apparent to one of ordinary skill in the art, the selection of these minors will vary depending on the physical and chemical characteristics of the particular ingredients selected to make the hair care composition.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of Embodiments of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A hair care composition for cleansing and refreshing the hair and the scalp comprising:
a) from about 0.1 wt. % to about 12 wt. % of a 1,2-diol having a carbon chain with a length of more than 8 carbons;
b) from about 0.1 wt. % to about 10 wt. % of a solid particle, wherein
(1) the interfacial tension between the solid particle and sebum is from about 5 to about 18 dyn/cm;
(2) the sebum exhibits spreading coefficient on the solid, which is greater than about 22 dyn/cm; and
(3) the work of adhesion of the sebum to the solid particle, which is greater than about 75 dyn/cm. from about 0.1 wt. % to about 5 wt. % emulsifier selected from the group consisting of anionic, non-ionic, cationic and amphoteric and mixtures thereof;
c) from about 0.005 wt. % to about 2 wt. % of a malodor protection material; and
d) an aqueous carrier.

2. A hair care composition according to claim 1, further comprises from about 0.1% to about 5% a detersive surfactant by weight of the hair care composition selected from the group consisting of anionic, non-ionic, cationic and amphoteric and mixtures thereof.

3. A hair care composition according to claim 1, further comprising a polymeric rheology modifier.

4. A hair care composition according to claim 1, further comprising one or more solvents.

5. A hair care composition according to claim 4, wherein the solvent is selected from the group consisting of 1,3-butanediol, isopropyl myristate, 2-hexyldecanol, propylene glycol, PEG-3 glyceryl cocoate, PEG-7 glyceryl cocoate, isododecane, isoparaffin C11-12 and mixtures thereof.

6. A hair care composition according to claim 1, wherein the solid particle of the hair care composition is selected from the group consisting of zinc carbonate, hydrophobically-modified silica, hydrophobically modified clay, zinc oxide, polyethylene powders, polypropylene powders, polystyrene powders, calcium silicate, nylon, boron nitride, mica, zeolite, cyclodextrins, fumed silica, synthetic clays, fluorocarbon resins, polypropylene modified starches of cellulose acetate, particulate cross-linked hydrophobic acrylate or methacrylate copolymers and mixtures thereof.

7. A hair care composition according to claim 1, wherein the 1,2-diol is selected from the group consisting of 1,2-dodecanediol, 1,2-decanediol, 1,2-octadecanediol and mixtures thereof.

8. A hair care composition according to claim 1, wherein the hair care composition is a leave-on composition.

9. A hair care composition according to claim 1, wherein the hair care composition is a rinse-off composition.

10. A hair care composition according to claim 1, wherein the hair care composition is delivered via a spray device.

11. A hair care composition according to claim 1, wherein the aqueous carrier is a combination of water and ethanol.

12. A hair care composition according to claim 1, wherein the malodor protection material is selected form the group consisting of an odor blocking material or an odor neutralizing material.

13. A hair care composition according to claim 1, wherein the malodor protection material is an odor masking material.

14. A hair care composition according to claim 1, wherein the malodor protection material is selected from the group consisting of methyl anthranilate, ethyl anthranilate, propyl anthranilate, isopropyl anthranilate, butyl anthranilate, isobutyl anthranilate, pentyl anthranilate, hexyl anthranilate, heptyl anthranilate, octyl anthranilate, menthyl anthranilate, geranyl anthranilate, phenethyl anthranilate, 2-acetamido benzoate, dimethyl anthranilate, methyl maltol, ethyl maltol, propyl maltol, dimethyl-3-(3-methylphenyl)-propanol, 2-methyl 2-pentenoic acid, and mixtures thereof.

15. A hair care composition according to claim 1, wherein the malodor protection material is selected from the group consisting of 2-methyl-2-pentenoic acid and ethyl maltol, methyl maltol and mixtures thereof.

16. A hair care composition according to claim 1, wherein the hair composition further comprises perfume.

17. A hair care composition according to claim 1 wherein the aqueous carrier is from about 80% to about 95% by weight of the hair care composition.

18. A hair care composition according to claim 1 wherein the aqueous carrier comprises from about 30% to about 60% by weight of the hair care composition ethyl alcohol.
